(19) United States
(12) Patent Application Publication
(10) Pub. No.: US 2024/0003746 A1
(43) Pub. Date: Jan. 4, 2024

(54) MULTIFUNCTIONAL SENSOR FOR MONITORING PREMISES AND METHODS USING SUCH A SENSOR

(71) Applicants: Khaled ABOUSALEH, SCHINDELLEGI (CH); ZYNNON AG, Schindellegi (CH)

(72) Inventor: Khaled ABOUSALEH, Schindellegi (CH)

(21) Appl. No.: 18/250,154

(22) PCT Filed: Oct. 14, 2021
(86) PCT No.: PCT/EP2021/078497

§ 371 Date: Jun. 15, 2023

(30) Foreign Application Priority Data
Oct. 23, 2020 (FR) 2010900

(51) Int. Cl. G01J 5/00; G01P 13/00; G01N 33/00; G01P 15/18

(52) U.S. Cl. CPC G01J 5/0025; G01P 13/00; G01N 33/0027; G01P 15/18

(57) ABSTRACT

Disclosed is a monitoring device including an infrared sensor and, furthermore, at least one sensor from barometric pressure, temperature, movement, sound level, relative humidity, ambient light, UV index, air quality and particulate sensors. Also disclosed is a system including such a device and a remote processor. Further disclosed are methods using such a device, particularly for monitoring a person, detecting the start of a fire or warning about a mechanical breakdown.

### CROSS-REFERENCE TO RELATED APPLICATIONS

This application is the U.S. national phase of International Application No. PCT/EP2021/078497 filed Oct. 14, 2021, which designated the U.S. and claims priority to FR 2010900 filed Oct. 23, 2020, the entire contents of each incorporated herein by reference.

### BACKGROUND OF THE INVENTION

### Field of the Invention

The present invention relates to the field of the monitoring of premises, or external volumes, which are mobile or immobile, and in particular to home automation.

### Description of the Related Art

Modern sensors do not make it possible to ascertain enough information to deduce a state of a location, of a person or of an object. They are even less suited to anticipating such a state, in order to deduce a possible, useful or necessary action.

The aim of the invention is to propose a device capable of collecting enough information about a location, a person or an object to ascertain a state thereof and to deduce a future state thereof if necessary.

According to a first subject of the invention, a preferably battery-operated monitoring device comprises an infrared sensor and also at least one sensor from among:
a barometric pressure sensor;
a temperature sensor;
a motion sensor, preferably a three-axis accelerometer;
a sound level sensor;
a relative humidity sensor;
an ambient light sensor;
a sensor for measuring a UV index;
a sensor for determining an air quality, preferably in order to measure a concentration of a substance and/or a CO2 equivalent therein;
a particle sensor, preferably for particles of a size between 0.3 μm and 10 μm;
   and/or
a preferably satellite-based geographical position sensor;
   and preferably:
a current sensor for analyzing and predicting a remaining life of the battery.

It is also possible to use a plurality of sensors of one and the same type. For example, it is possible to use a plurality of infrared sensors. It is also possible to use a plurality of temperature or humidity sensors that have different operating ranges.

The sensor for determining air quality may comprise means for measuring a concentration of one of the substances from among ethanol and dihydrogen, or a total amount of volatile organic compounds.

The device is preferably battery-powered. This is an advantage over existing devices on the market that require installing a power cable connected to the mains, and therefore makes it possible to achieve easy installation of the device in locations where there is no wired electric power supply.

Advantageously, the device comprises wireless transmission means for outgoing data, for example cellular, Bluetooth and/or Wi-Fi transmission means, thereby making it possible to have flexible installation. The device is able to send data in real time or store said data so as to send them later, at regular intervals or not, automatically or upon request. Preferably, the device is also intended to receive incoming information using the same means. These data may be specific configuration data or an update to software embedded in the device.

The device may have a display for the temperature measured using the infrared sensor.

The infrared sensor may comprise means for measuring a temperature in a plurality of sectors, independently, of one and the same observation cone.

According to a second subject, the invention relates to a method for detecting the presence of a person, using a system according to the invention and comprising a device according to the invention having means for measuring a temperature in a plurality of sectors, wherein the presence is deduced from the temperatures measured in each sector.

According to a third subject, the invention relates to a method for measuring a temperature of a person, using a system according to the invention and comprising a device according to the invention having means for measuring a temperature in a plurality of sectors, comprising measuring a temperature of the person in a first sector and simultaneously measuring an ambient temperature in a second sector with the infrared sensor and calculating a temperature of the person by correcting the measured temperature of the person on the basis of the measured ambient temperature, and preferably also on the basis of data from at least one of the following sensors: temperature sensor, relative humidity sensor, ambient light sensor and UV index sensor.

According to a fourth subject, the invention relates to a method for providing a fire alarm, using a device according to the invention, which comprises steps of:
monitoring the presence of a person;
   and monitoring at least one of the following measurements:
a temperature increase above a given threshold;
an increase in the CO2 level above a given threshold;
an increase in brightness above a given threshold;
a decrease in humidity below a given threshold;
   and, when one of said thresholds is crossed, triggering said fire alarm, and, if the device comprises a geographical position sensor, transmitting the geographical position coordinates to an emergency service.

According to a fifth subject, the invention relates to a method for evaluating a sleep quality of a person, using a device according to the invention, which comprises steps of:
detecting the presence of a person;
   and simultaneously collecting at least one of the following data:
sound levels;
temperatures;
humidity;
atmospheric pressure;
brightness;
air quality,
   and then cross-correlating these data.

According to a sixth subject, the invention relates to a method for preventing a risk of hypothermia for a person, using a device according to the invention and comprising steps of:
detecting the presence of a person;
monitoring a temperature of said person;
monitoring a sound level;
   and, when the temperature of the person and the sound level decrease below given respective thresholds, triggering an acoustic alarm to wake up the person.

According to a seventh subject, the invention relates to a method for warning of a risk of failure of a machine, using a device according to the invention and comprising steps for carrying out:
temperature measurements on moving parts, using the infrared sensor;
ambient temperature measurements;
sound level measurements; and, preferably
humidity measurements; and/or
acceleration/deceleration and vibration measurements.

### BRIEF DESCRIPTION OF THE DRAWINGS

A number of embodiments of the invention will be described below, by way of non-limiting example, with reference to the appended drawings, in which:

FIG. 1 is a perspective schematic view of a room in which a monitoring device according to the invention is installed, said monitoring device in particular being equipped with an infrared sensor, and illustrating an observation cone of this infrared sensor;

FIG. 2 is a perspective schematic view of the device of FIG. 1, in isolation;

FIG. 3 is an elevated schematic view of what is seen by the infrared sensor and of a division into forty-nine sectors of the area under observation; and

FIG. 4 is a diagram illustrating an interpretation, by the device, of the area under observation, for each of the forty-nine sectors of FIG. 3.

### DETAILED DESCRIPTION

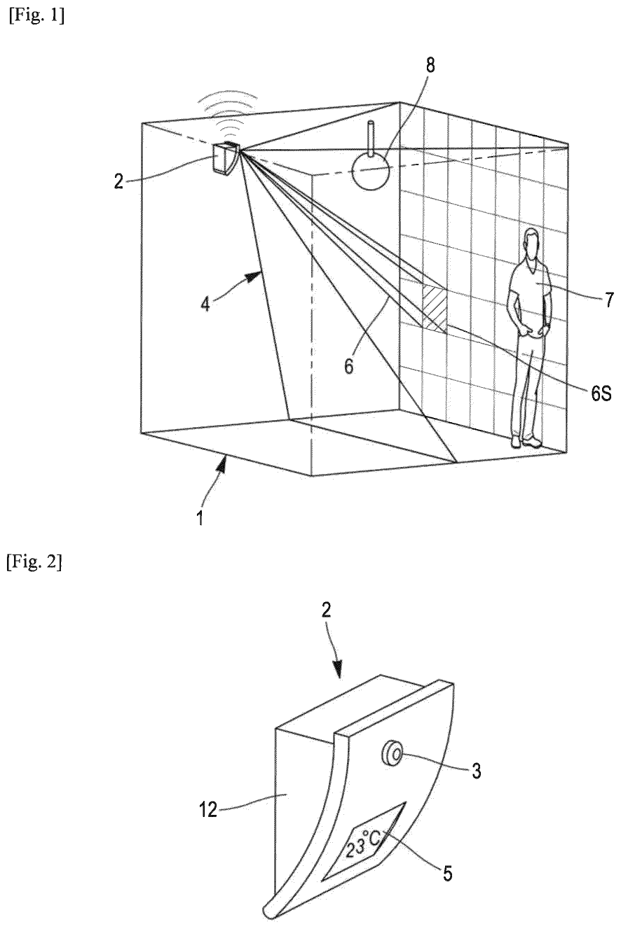

FIG. 1 shows a volume, here a room 1, monitored by a device 2 according to the invention. The device 2 comprises an infrared radiation sensor 3 visible on a front face of the device in FIG. 2. As illustrated in FIG. 1, the infrared sensor 3 scans an observation cone 4, divided into sectors 6, with a substantially rectangular cross section 6S. The device 2 comprises display means 5, advantageously intended to display thereon a temperature measured by the infrared sensor.

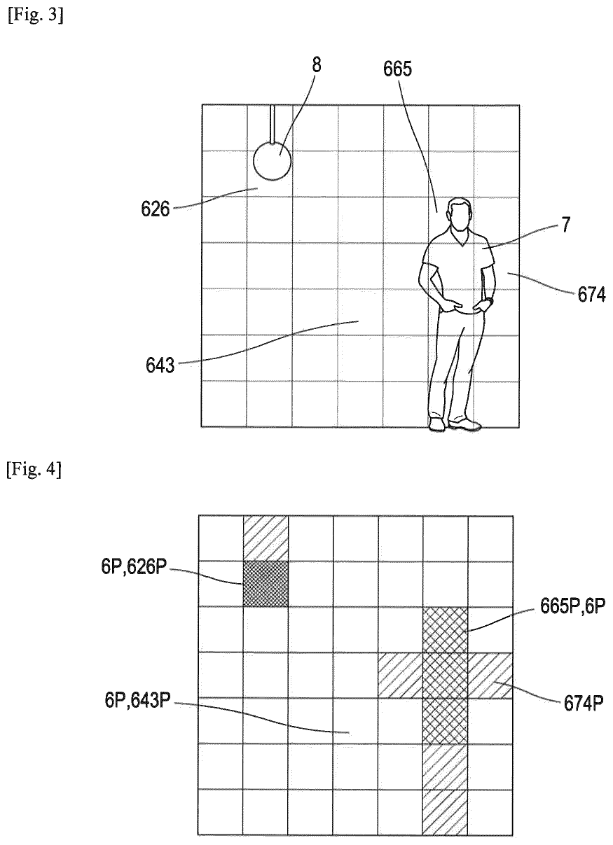

FIG. 3 illustrates a portion of what is seen by the infrared sensor 3 in forty-nine of the sectors 6 of the cone 4. The forty-nine sectors 6 are distributed into seven vertical columns and seven vertical rows.

FIG. 4 illustrates the interpretation given by the infrared sensor 3 of what is seen and illustrated in FIG. 3. The room 1 contains a person 7 and a luminous globe 8. The person is standing and the globe is suspended from the ceiling of the room. The sensor 3 measures the infrared radiation emitted in each of the sectors 6. FIG. 4 illustrates, for each of the sectors 6, a digitized representation of what is seen. Thus, for each sector 6, the device measures an average temperature, represented by a darker or lighter pixel 6P in the figure.

Thus, for example:
a very light pixel 643P, corresponding to the sector 643, measures an ambient temperature of the room 1;
a very dark pixel 626P, corresponding to the sector 626, incorporating the majority of the globe 8, measures a high temperature;
pixels 665P and 674P, which are more or less dark, each correspond to a larger or smaller part of the body of the person 7.

In a housing 12, the device 2 furthermore comprises:
a barometric pressure sensor
a motion sensor (3-axis accelerometer)
a sound level sensor
a relative humidity sensor
an ambient light sensor
a sensor for measuring a UV index
an air quality sensor, that is to say a sensor for detecting and measuring a concentration of volatile organic compounds or a CO2 equivalent. The volatile compounds may in particular comprise ethanol and hydrogen (H2),
a satellite-based geographical position sensor.

In the illustrated example, the device 2 is battery-powered, or powered by a cell, in order to be able to be installed easily in any location without a connection. It comprises a current sensor that makes it possible to analyze and to predict the lifetime of the battery.

A description will now be given of a plurality of methods that may be implemented using a system comprising a device 2 according to the invention. These methods generally comprise prior learning using an artificial intelligence. The system may comprise one or more remote computing means for remotely processing data supplied by the device 2.

One method makes it possible to detect, or assume, the presence of a person, primarily with the aid of the infrared sensor, in particular by analyzing the pixels 6P, the temperature that is revealed, a number and a distribution of the pixels, their change, which may correspond to the movement of a person. This detection method is perfectly suited to learning using an artificial intelligence.

One method makes it possible to measure the temperature of a human body. It is preferably combined with the above method for detecting the presence of a person. The main sensor that is used to measure the temperature of the human body is the infrared sensor 3. The temperature of the human body may vary according to a plurality of parameters outside the human body, in particular ambient temperature, ambient humidity, ambient light level and ambient UV (ultraviolet) index. Taking only a measurement from the infrared sensor as a basis to ascertain the temperature of the human body may lead to an erroneous value. In order to make a correction to the infrared-based human body temperature measurement and have a more exact and precise value, the direct measurement of the infrared sensor may be carried out in particular based on a value measured in the sector 665; it is corrected based on data supplied by other sensors, in particular the ambient temperature sensor, the relative humidity sensor, the ambient light sensor and the UV index sensor, and it is also corrected by a measurement of the ambient temperature, for example measured in the sector 643. The measurements and information supplied by the various sensors at the same time and at the same location make it possible to supply a more reliable measurement of the temperature of the human body. Such a result is made possible by the combined use of the various sensors.

The device 2 is intended to operate autonomously and automatically. However, the infrared sensor has known and specific usage limits. These limits depend on ambient temperature, ambient humidity, atmospheric pressure, light incidence and UV level. In order to guarantee correct usage operation of the product, the measurements from various sensors, in particular from the ambient temperature sensor, the relative humidity sensor, the ambient light sensor and the UV index sensor, are used in order to guarantee that the infrared sensor operates within predefined operating ranges, within limits. This guarantees and safeguards optimum efficiency of the measurements carried out by the infrared sensor. Thus, a method for automatically controlling the operation of the infrared sensor 3 is carried out in real time by the device 2, by virtue of the simultaneous use of a plurality of the sensors included in this device.

The temperature of the human body depends on the ambient temperature and the ambient humidity; taking into account these parameters, the device supplies a more precise measured temperature of the human body.

A method for providing an alarm may also be implemented using a device according to the invention.

The infrared sensor makes it possible to detect the presence of people in its field of view. In the case of a fire starting, when the device detects an increase in temperature above a given threshold and/or an increase in a level of carbon dioxide or carbon monoxide above a given threshold and/or an increase in brightness and/or a drop in humidity, in combination with the detection of the presence of a person in the monitored volume, this makes it possible to trigger a smart alarm. Indeed, it may allow the emergency services to intervene more quickly at the location where the presence of a person has been detected. Furthermore, the GPS coordinates sent with the alarm make it possible to quickly locate the location of the fire. The device according to the invention is therefore more efficient than a CO2 sensor on its own or a temperature sensor on its own. Furthermore, the infrared sensor detecting a heat source is capable of detecting the start of a fire even before the fire or carbonic gas is present. The carbonic gas sensor then makes it possible to confirm that a fire has actually started. All of the information supplied by the sensors of the device gives the fire service an early alarm with complete information that allows a more effective and faster intervention.

A method for evaluating the quality of sleep of a person may also be implemented using the device 2 by combining the information from a plurality of the sensors.

The infrared sensor identifies the presence of a person by measuring their temperature, the sound level sensor measures the snoring level of the person, and the temperature, humidity, pressure, light level and air quality sensors determine the state of the environment. These combined measurements make it possible to supply complete information about the sleep quality of the person and the links that may exist between this sleep quality and the state of the environment.

A method for providing an alert in the event of hypothermia for a person may also be implemented using the device 2 by combining the information from a plurality of the sensors.

Diabetic people exhibit risks of hypothermia while they are sleeping. Their temperature may drop without them realizing. The presence of the infrared sensor, by monitoring the temperature at regular intervals, may trigger an acoustic alarm when the temperature drops below a certain value in order to wake up the person.

A method for monitoring the state of health of a person may also be implemented using the device 2.

The device is thus able to measure both the temperature of the body of the person and the sound level in the room. A high measurement of the temperature in combination with an acoustic measurement of coughing may provide a significant indication about the state of health of the person, and about the development thereof over time.

A method for monitoring the pregnancy of a person may also be implemented using the device 2.

The body temperature of a female changes throughout the menstrual cycle. In the course of a normal cycle, during the follicular phase, it is at an initial value lower than 37° centigrade and does not vary much. Just before ovulation, the temperature drops: it is at the lowest point in the cycle. It then increases again above 37° C. It stays at this level throughout the duration of the luteal phase, for the last twelve or fourteen days of the menstrual cycle. In the event of pregnancy, the thermal plateau lasts beyond 28 days. By contrast, if fertilization has not taken place, the temperature drops back to its initial level, just before the period arrives. The device is thus able to continuously measure the body temperature of a female and thus makes it possible to provide an indication of pregnancy.

An alertness method, in particular for a person living alone, more specifically for an elderly person, may also be implemented using the device 2.

The device measures the temperature of a person and thus determines their presence, but also their mobility. The device is thus able to determine whether a person is immobilized. In the event of prolonged immobilization and in the event of a significant reduction in sound level, the device may for example conclude that the person has fallen or is ill and trigger an alarm or contact another person, preferably a designated person. The device, in particular by virtue of its sound sensor, may also be programmed to recognize an “emergency word” intended to be spoken by the single person when they need urgent assistance, for example in the event of a fall.

The device 2 may be mobile. It may in particular be installed in a transport vehicle, for example a bus or a train.

When the device is installed in a public transport vehicle, it may be integrated into a transport management system and method. It may thus make it possible to measure the temperature of people entering the vehicle. It makes it possible to filter out people having a temperature that exceeds a certain threshold, to count the number of people entering the vehicle and to link this number of people to a geographical position of the vehicle. The device may therefore make it possible to implement a transport management method that provides a real-time occupancy level in public transport vehicles in real time, and/or a level of filtering out by temperature and/or a geographical coverage of these figures.

A plurality of devices according to the invention may be networked. They may in particular be connected to a cloud, that is to say a cloud network.

With the sensors being connected to the cloud, the measurements supplied by each device may be correlated in order to supply information about a given geographical area.

A plurality of devices indicating an increase in CO2 concentration make it possible to supply information about pollution in a given geographical area and the direction of movement of this pollution.

A plurality of devices supplying vibration measurements make it possible to give information relating to an earthquake, and may make it possible to determine the epicenter thereof and the amplitude thereof at a given location.

It is thus possible to determine, in a given area:
a map of sound levels;
a map of pressure, temperature and humidity level change; or
a map of light or sunlight levels.

A sensor according to the invention may also be used in industry, in particular to implement a method for anticipating failure of a machine.

It is possible to install this device, or a plurality of these devices, in or close to a machine in operation; it may then transmit:
temperature measurements on moving parts, taken by the infrared sensor;
ambient temperature measurements;
sound level measurements;
humidity measurements;
acceleration/deceleration and vibration measurements; and/or
other data supplied by the sensors of the device.

The data may be stored in the cloud and learning algorithms (machine learning) are applied in order to establish the link between a failure and the data from the sensors. A system according to the invention, thus applied, makes it possible to predict machine failures. By way of example, an increase in temperature, vibration level and sound level makes it possible to conclude as to a failure in the near future; the type of vibration may also make it possible to identify a defective component.

In the illustrated example, to use infrared to measure the temperature, it is possible to use an electronic component of the type D6T-44L-06, supplied by Omron.

Of course, the invention is not limited to the examples that have just been described. On the contrary, the invention is defined by the following claims.

Indeed, it will be apparent to those skilled in the art that various modifications may be made to the embodiments described above in light of the teaching that has just been disclosed to them.

A plurality of methods may in particular be implemented in parallel by one and the same device. These methods may be from among those described above, or others that are not described. For example, one and the same sensor may serve both to detect fire and to warn of a risk of hypothermia and a risk of falling.

A device according to the invention may have numerous applications, in particular:
a. For a smart home:
   i. measuring comfort in the home, which is particularly important for elderly people;
   ii. regulating heating;
   iii. detecting indoor air pollution;
   iv. measuring sunlight and brightness, thereby making it possible to automatically open and close blinds;
   v. detecting the presence or the absence of a person;
   vi. providing an alarm;
   vii. monitoring the movement of people, in particular elderly people, and alerting of a fall when a person is no longer moving;
   viii. continuously measuring, in real time, the temperature of people and detection in the event of a threshold being exceeded, this being beneficial in particular for elderly people and children;
   ix. measuring and monitoring sound level;
   x. safety in the event of excessive heat or cold;
   xi. early detection of earthquakes, by measuring vibrations not able to be detected by human beings, which may make it possible for example to automatically actuate gas valve closure mechanisms and to disconnect an electric power supply; and
   xii. early detection of a heat source that could cause a fire.
b. For a school, a restaurant or any public establishment:
   i. everything indicated above with reference to a home
   ii. automatically monitoring individual temperatures of people entering enclosed locations and detection in the event of a threshold being exceeded, in particular to limit transmission of viruses in enclosed environments.
c. In public transport, in particular on a bus or train:
   i. automatically monitoring individual temperatures of people entering
   ii. monitoring indoor air pollution
   iii. ascertaining the occupancy of transport in real time (the link may be established with the geographical position of the vehicle)
   iv. measuring vibrations of the vehicle
   v. measuring temperature and humidity in order to regulate heating or air conditioning
   vi. evaluating the driving of drivers by measuring accelerations and decelerations
   vii. tracking the position of the vehicle in real time
   viii. fleet management and optimization
d. For hospitals and medical practices:
   i. everything indicated above with reference to schools
   ii. improving the safety of carers by identifying people with a fever
e. For airports:
   i. automatically monitoring individual temperatures of people
f. In shops and supermarkets:
   i. everything indicated above with reference to schools;
   ii. ascertaining how full shelves are in real time.
g. In offices:
   i. everything indicated above with reference to schools
   ii. continuously monitoring temperature for people in “open space” offices
h. For building access systems:
   i. automatically monitoring individual temperatures of people entering a building.

A device according to the invention also has numerous advantages over existing products:
a. The products from the prior art use power supply cables, this making installation tricky and complex and limiting use to locations where it is possible to use a cable. The device using a battery is thus far more flexible in terms of installation.
b. The dimensions and the weight of the product are far less than those of products from the prior art, thereby allowing easier installation where necessary. The device may weigh less than 300 g and have dimensions smaller than 100×100×100 millimeters.
c. The device is able to be manufactured at a cost that may be twenty-five to thirty times lower than that of products from the prior art.
d. Few people are needed to operate a device and a system according to the invention: the operation of devices from the prior art generally requires at least two people: one person next to the appliance and one person in front of the screen to monitor a video image in which the temperature is displayed. A device according to the invention does not require any more than one person; it does not require an external screen or a person to monitor such a screen.
e. A device according to the invention does not capture any image or video, and it therefore respects privacy. It also does not provide any possibility of facial recognition. The products from the prior art capture videos displaying the temperature. These videos may be recorded and allow facial recognition of people.
f. A device according to the invention has a plurality of sensors that take physical measurements at one and the same time, thereby making it possible to perform very powerful correlation analysis between the various phenomena, for example a link between pollution and a temperature. This also allows the use of an artificial intelligence algorithm for finding complex correlations between various physical data for a given application.
g. A system according to the invention may comprise a network connection, Internet connection or cloud connection, which facilitates analysis of the collected data. Thus, besides a body temperature supplied and displayed instantaneously, all of the other measurements may be sent to a cloud, thereby allowing remote analysis of the data. Depending on the application, with one and the same device according to the invention, it is possible to use a separate data analysis program. Likewise, services provided based on the data analysis may be highly different from one application to another.

1. A monitoring device, comprising:
an infrared sensor, and
a temperature sensor.

2. The device as claimed in claim 1, further comprising a barometric pressure sensor.

3. The device as claimed in claim 1, further comprising a motion sensor.

4. The device as claimed in claim 1, further comprising a sound level sensor.

5. The device as claimed in claim 1, further comprising a relative humidity sensor.

6. The device as claimed in claim 1, further comprising an ambient light sensor.

7. The device as claimed in claim 1, further comprising a sensor for measuring a UV index.

8. The device as claimed in claim 1, further comprising a particle sensor.

9. The device as claimed in claim 1, further comprising a sensor for determining an air quality.

10. The device as claimed in claim 1, further comprising a plurality of sensors of one and the same type.

11. The device as claimed in claim 1, wherein the infrared sensor comprises means for measuring a temperature in a plurality of sectors, independently, of one and the same measurement cone.

12. A monitoring system comprising remote processing means and the device as claimed in claim 1, said device furthermore comprising means for wireless communication with said remote processing means.

13. A method for detecting the presence of a person, using a system comprising:
remote processing means, and
a first said device as claimed in claim 1, said device furthermore comprising means for wireless communication with said remote processing means, and
a second said device as claimed in claim 1, wherein the infrared sensor comprises means for measuring a temperature in a plurality of sectors, independently, of one and the same measurement cone,
wherein said method comprises deducing the presence of the person from temperatures measured in each said sector.

14. A method for measuring a temperature of a person, using a system comprising:
remote processing means, and
a first said device as claimed in claim 1, said device furthermore comprising means for wireless communication with said remote processing means, and
a second said device as claimed in claim 1, wherein the infrared sensor comprises means for measuring a temperature in a plurality of sectors, independently, of one and the same measurement cone,
the method comprising:
measuring a temperature of the person in a first sector and simultaneously measuring an ambient temperature in a second sector with the infrared sensor, and comprises
calculating a temperature of the person by correcting the measured temperature of the person on the basis of the measured ambient temperature and on the basis of data from at least one of the sensors from among: a temperature sensor, a relative humidity sensor, an ambient light sensor, and a UV index sensor.

15. The monitoring device of claim 1, wherein the device is battery-operated and further comprises a current sensor configured to analyze and predict a remaining life of the battery.

16. The monitoring device of claim 15, further comprising a satellite-based geographical position sensor.

17. The device of claim 3, wherein the motion sensor is a three-axis accelerometer.

18. The device of claim 8, wherein the particle sensor is capable of sensing particles of a size between 0.3 μm and 10 μm.

19. The device of claim 9, wherein the sensor for determining air quality is configured to measure a concentration of a substance and/or a CO2 equivalent therein.

20. The monitoring device of claim 1, further comprising:
a satellite-based geographical position sensor;
a current sensor constructed to analyze and predict a remaining life of the battery;
a barometric pressure sensor;
a motion sensor;
a sound level sensor;
a relative humidity sensor;
an ambient light sensor;
a sensor for measuring a UV index;
a particle sensor; and
a sensor for determining an air quality;
wherein the infrared sensor comprises means for measuring a temperature in a plurality of sectors, independently, of one and the same measurement cone.
